(19) United States
(12) Patent Application Publication
(10) Pub. No.: US 2015/0225507 A1
(43) Pub. Date: Aug. 13, 2015

(54) ELASTOMERIC POWDERS

(71) Applicant: DOW CORNING CORPORATION, Midland, MI (US)

(72) Inventors: Geoffrey Debaugnies, Masnuy-Saint-Jean (BE); Sabrina Salvati, Flenu (BE); Marc Thibaut, Arquennes (BE)

(73) Assignee: Dow Corning Corporation a corporation

(21) Appl. No.: 14/419,843

(22) PCT Filed: Aug. 7, 2013
(86) PCT No.: PCT/GB2013/052110

§ 371 Date: Feb. 5, 2015

(30) Foreign Application Priority Data
Aug. 7, 2012 (GB) 1214115.6

(51) Int. Cl. C08G 77/38; A61Q 19/00; A61K 8/895; C09D 183/04

(52) U.S. Cl. CPC C08G 77/38; C09D 183/04; A61Q 19/00; A61K 8/895; A61K 2800/805; A61K 2800/10

(57) ABSTRACT

A method for preparing an elastomeric silicone powder comprises the steps of: (i) forming a mixture of organopolysiloxane A and organopolysiloxane B, wherein organopolysiloxane A comprises more than 0.5% alkenyl groups by weight of the organopolysiloxane A, organopolysiloxane B comprises from 0.25% to 1% silicon-bonded hydrogen atoms by weight of the organopolysiloxane B; and (ii) spray curing the mixture of step (i) to form an elastomeric silicone powder.

### BACKGROUND OF THE INVENTION

Disclosed herein is a method for preparing elastomeric silicone powders and applications of said elastomeric silicone powders, for example in coatings and in personal care applications.

When used in coating applications (for example, on surfaces such as wood, or automotive interiors), elastomeric silicone powders can provide a number of advantageous characteristics to the surfaces on which they are applied; for example: flatness, smoothness, anti-slip, anti-scratch, mattifying without opacity.

When used in personal care applications, elastomeric silicone powders may provide personal care products with a soft touch and thickening properties.

The aforementioned properties are derived from the elastomeric nature of the silicone powders (ie the ability of the particles of the powder to be deformable, but to resume their original shape after the deforming force is removed), and from the relatively small particulate structure that is inherent of a powder.

There exist various methods to make elastomeric silicone powders in the art. Some require the use of predispersion of raw materials in solvents, which subsequently need to be evaporated. Other existing processes rely on curing processes; such as addition-reaction processes, organoperoxide-based radical reaction-curing processes, and condensation-reaction processes. The curing process used to form elastomeric silicone powders may be carried out as a spray curing process, which involves the spraying of the starting materials in a substantially liquid form into a curing chamber with the application of heat (at least at the point of entry to the chamber). However, it has been found that conventional spray curing processes used to form elastomeric silicone powders result in a significant deposit of silicone on the walls of the spray curing chamber. This deposit builds up over repeated use of the chamber, resulting in the need to periodically stop the process in order to enable a cleaning of the walls of the chamber. The deposit may also occlude pipes and nozzles of the spray curing apparatus. These deposits lead to a poor recovery of elastomeric silicone powder particles, and loss of materials and/or man hours.

### BRIEF SUMMARY OF THE INVENTION

Disclosed herein is a method for preparing elastomeric silicone powders and applications of said elastomeric silicone powders, for example in coatings and personal care applications.

The method allows for a relatively low deposit of elastomeric silicone on the walls of the spray curing chamber, and so compensates for a number of deficiencies discussed above.

### DETAILED DESCRIPTION OF THE INVENTION

Accordingly, in a first aspect of the present invention, there is provided a method for preparing an elastomeric silicone powder comprising the steps of:
(i) forming a mixture of organopolysiloxane A and organopolysiloxane B, wherein organopolysiloxane A comprises more than 0.5% alkenyl groups by weight of the organopolysiloxane A, organopolysiloxane B comprises from 0.25% to 1% silicon-bonded hydrogen atoms by weight of the organopolysiloxane B, and;
(ii) spray curing the mixture of step (i) to form an elastomeric silicone powder.

The method has been demonstrated to result in less than 0.5%, less than 0.1%, less than 0.05%, or substantially none of the mixture formed in step (i) being deposited on the walls of the spray curing chamber in which step (ii) is practiced.

The method has been demonstrated to produce elastomeric silicone powders in the form of particles, or agglomerates of particles, with an average diameter of 0.1 to 1000 μm, alternatively 1 to 500 μm, alternatively 1 to 200 μm, alternatively 1 to 100 μm.

All viscosities referred to herein are taken at 25° C. and standard atmospheric pressure (i.e. 101.325 kPa), unless otherwise indicated.

Spray curing is assumed to be understood by the man skilled in the art, but may mean a reaction where the reactants are provided in a liquid form (possibly a liquid mixture), and forced into an atomized state, in a spray curing tank where the curing takes place, without the need to evaporate a solvent, e.g. water. The atomized state is reached by spraying or vaporising the liquid mixture in the curing tank at a temperature ranging of from +20 to +350° C., alternatively of from +80° C. to +350° C., alternatively of from +80° C. to +250° C.

Spray drying is assumed to be understood by the man skilled in the art, but may mean a process where a solvent such as water is removed by atomization of a dispersion or emulsion. The spray drying process should not involve any particular chemical reaction.

Organopolysiloxanes may generally comprise various siloxy units such as (i) (R′₃SiO_{1/2})_{a′}, (ii) (R′₂SiO_{2/2})_{b′}, (iii) (R′SiO_{3/2})_{c′}, or (iv) (SiO_{4/2})_{d′}, units which are commonly known in the art, and also used herein, as M, D, T, and Q units respectively, wherein R′ is a monovalent organic group; a′, b′, c′ and d′ have a value of from 0 to 0.9, with the proviso that the value of a′+b′+c′+d′=1.

Monovalent organic groups include hydrocarbons such as alkyl groups (e.g. methyl, ethyl, propyl, isopropyl, butyl, octyl, nonyl, tetradecyl and/or octadecyl); cycloalkyl groups (e.g. cyclohexyl and/or cycloheptyl); alkenyl groups (e.g. vinyl and/or hexenyl); aryl groups (e.g. phenyl, diphenyl and/or naphthyl); alkaryl groups (e.g. tolyl, xylyl and/or ethylphenyl); aralkyl groups (e.g. benzyl and/or phenylethyl); or any mixture thereof.

The monovalent organic group may contain substituents other than carbon and hydrogen atoms, such as halogen atoms (chlorine, fluorine, bromine, iodine); halogen atom containing groups such as haloalkyl groups (chloromethyl, perfluorobutyl, trifluoroethyl, and nonafluorohexyl) and haloaryl groups (monochlorophenyl, dibromophenyl, tetrachlorophenyl, monofluorophenyl); oxygen atoms; oxygen atom containing groups such as hydroxy, carboxyl, carbinol, ester, ether, acrylic groups and polyoxyalkylene groups (polyoxyethylene, polyoxypropylene, polyoxybutylene); nitrogen atoms; nitrogen atom containing groups such as nitrile, amino, amido, cyano, cyanoalkyl and urethane groups; sulphur atoms; sulphur atom containing groups such as sulphide, sulphone, sulphate, sulphonate and mercapto groups; phosphorus atoms; phosphorus atom containing groups such as phosphate, phosphate and phosphonate groups; or any mixture thereof.

The letter designations M, D, T, and Q, refer respectively, to the fact that the siloxy unit is monofunctional, difunctional, trifunctional, or tetrafunctional. These M, D, T, and Q structural units are depicted below for the sake of clarity

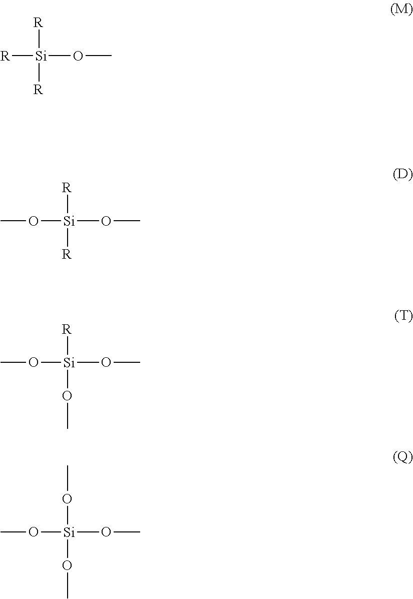

Organopolysiloxanes A and/or B may be cyclic, linear, branched, or crosslinked. A cyclic organopolysiloxane would be considered to be one that includes predominantly or only D-units and optionally T units, but typically no M unit in the ring. A linear organopolysiloxane would be considered to be one that includes predominantly D units and which is terminated by M units. A branched organopolysiloxane would be considered to include at least 1 T or at least 1 Q unit, along the chain or at terminal positions. Organopolysiloxane resins are examples of branched organopolysiloxanes, where more than 30% of siloxy units, alternatively more than 80% siloxy units, are either T or Q units.

The Organopolysiloxane A may have the alkenyl groups in terminal or pendant positions or both. Organopolysiloxane A has at least 2 alkenyl groups. The alkenyl group may be linear or cyclic, composed of from 2 to 10 carbon atoms, alternatively of from 2 to 6 carbon atoms, such as hexenyl, vinyl, allyl or pentenyl or cyclohexenyl. Organopolysiloxane A may be any combination of two or more of organopolysiloxanes each comprising at least 2 alkenyl groups.

Organopolysiloxane A may be an MQ resin consisting of units of the general formula SiO_{4/2} and R^{q}₃SiO_{1/2} wherein at least 2 R^{q} substituents are alkenyl groups and the remainder are alkyl groups.

Suitable organopolysiloxanes A may be of the Q-branched structure, that is including at least 1 Q unit and further D and M units, optionally T units, such as (MD₎₄Q organopolysiloxanes.

Organopolysiloxanes A may conform to Formula I, comprising more than 0.5% alkenyl groups by weight of the organopolysiloxane A:

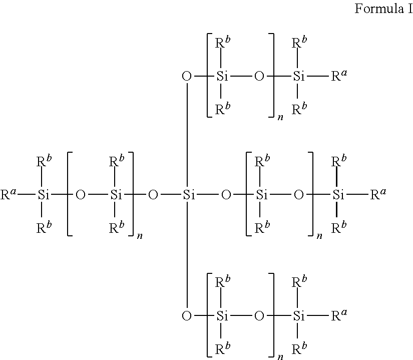

wherein each R^{a} substituent is an alkyl group having 1-6 carbon atoms, an alkenyl group having 2-6 carbon atoms, or an alkynyl group having 2-6 carbon atoms; each Rb substituent is an alkyl group having 1-6 carbon atoms, an alkenyl group having 2-6 carbon atoms, an aryl group, an alkoxy group, an acrylate group, or a methacrylate group; n is 1-200; and provided that at least 3.2-3.9 of the Ra substituents are alkenyl or alkynyl groups. Further details of Q-branched organopolysiloxanes according to Formula I and that comprise more than 0.5% alkenyl groups by weight are described in International Patent Publication No. WO/2006/055233, each of which may be an organopolysiloxane A of the present invention. The organopolysiloxane A may be selected from those Q-branched organopolysiloxanes discussed above and that have a viscosity of ≦400 mm²/s. Consequently, the disclosures of such organopolysiloxanes described in International Patent Publication No. WO/2006/055233 is incorporated herein by reference.

Organopolysiloxane A may conform to the general formula

R^{c}R_{d}₂SiO(R^{d}₂SiO)_{x′}(R^{d}R^{e}SiO)_{y′}SiR^{d}₂R^{c}

where each Rd denotes an alkyl or cycloalkyl group having from 1 to 10 carbon atoms, for example, methyl, ethyl, propyl, butyl or cyclohexyl; each R^{c} and R^{e} denotes an alkenyl group or an alkynyl group; and x′ and y′ are such that Organopolysiloxane A has a viscosity at 25° C. of ≦400 mm^{2/}s.

Organopolysiloxane A may comprise from 0.5 to 15%, 0.5 to 10%, 0.5 to 5% alkenyl groups by weight of organopolysiloxane A. Organopolysiloxane A may comprise 0.8% alkenyl groups by weight of organopolysiloxane A.

The Organopolysiloxane B comprising silicon-bonded hydrogen atoms may be a cyclic, linear, or branched organopolysiloxane, or any mixture thereof. Organopolysiloxane B may be any combination of two or more of organopolysiloxanes comprising a silicone-bonded hydrogen atom.

Organosiloxane B may be selected from those according to Formula II, comprising of from 0.25% to 1% silicon-bonded hydrogen atoms by weight of the organopolysiloxane B,

[R^{f}₂HSiO_{1/2}]_{k′}[R^{f}₃SiO_{1/2}]_{r′}[R^{f}HSiO_{2/2}]_{m′}[R^{f}₂SiO_{2/2}]_{s′}[R^{f}SiO_{3/2}]_{p′}[SiO_{4/2}]_{q′} Formula II

where R^{f} may designate a univalent organic group that does not contain aliphatic
unsaturation; k′≧0;r′≧0; 1≧k′+r′≦10; 30 ≦m′≦200; 10 s′≦100; 0 ≦p′≦10; q′=1; and (k′+r′+m′+s′+p′+q′) is such that the viscosity of organopolysiloxane B is ≦100 mm²/s.

Further details of organopolysiloxanes B according to Formula II and that comprise of from 0.25% to 1% silicon-bonded hydrogen atoms by weight are described in Japanese Patent Application No. 2007211186 A, each of which may be organopolysiloxane B of the present invention, such that the viscosity of Organopolysiloxane B ≦100 mm²/s. Consequently, the disclosures of such organopolysiloxanes described in Japanese patent Application No. 2007211186 are incorporated herein by reference.

Organopolysiloxane B may have the general formula:

R^{g}₃SiO_{1/2}((CH₃)₂SiO_{2/2})_{e′}(R^{g}₂SiO_{2/2})_{f′})SiO_{1/2}R^{g}₃

where each R^{g} may be independently selected from an alkyl group having 1 to 4 carbon atoms or hydrogen, e′ is 0 or an integer, f′ is an integer such that e′+f′ is such that the viscosity of Organopolysiloxane B≦100 mm²/s.

Organopolysiloxane B may be selected from organohydrogenpolysiloxanes comprising at least one cyclosiloxane, according to Formula III, comprising from 0.25% to 1% silicon-bonded hydrogen atoms by weight of the organopolysiloxane B

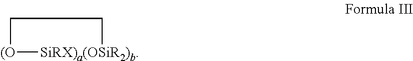

where each R is independently selected from a hydrogen atom and a monovalent hydrocarbon group comprising 1 to 20 carbon atoms which is free from aliphatic unsaturation, a is an integer from 1 to 18, b is an integer from 1 to 19, a+b is an integer from 3 to 20, each X is an independently selected functional group selected from a halogen atom, an ether group, an alkoxy group, an alkoxyether group, an acyl group, an epoxy group, an amino group, or a silyl group, or a —Z—R^{h} group, where each Z is independently selected from an oxygen and a divalent hydrocarbon group comprising 2 to 20 carbon atoms, each R^{h} group is independently selected from —SiRᵥY₃₋ᵥ , or a group described by formula (IV):

(Y₃₋ᵤRᵤSiO_{1/2})_{c}(Y₂₋ₒRₒSiO_{2/2})_{d}(Y₁₋ₚSiO_{3/2})ₑ(SiO₄₋₂)_{f}(CR_{q}Y_{1-q})_{g}(CRᵣY₂₋ᵣ)ₕ(O(CRₛY₂₋ₛ)ᵢ(CRₜY₃₋ₜ)ⱼ Formula IV:

where each R is as described above, the sum of c+d+e+f+g+h+i+j is at least 2, u is an integer from 0 to 3, o is an integer from 0 to 2, p is an integer from 0 to 1, q is an integer from 0 to 1, r is an integer from 0 to 2, s is an integer from 0 to 2, t is an integer from 0 to 3, v is an integer from 0 to 3, each Y is an independently selected functional group selected from a halogen atom, an ether group, an alkoxy group, an alkoxyether group, an acyl group, an epoxy group, an amino group, a silyl group, or a Z-G group, where Z is as described above, each G is a cyclosiloxane described by formula (V):

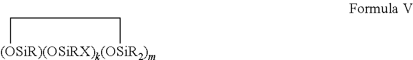

where R and X are as described above, k is an integer from 0 to 18, m is an integer from 0 to 18, k+m is an integer from 2 to 20, provided in formula (IV) that one of the Y groups is replaced by the Z group bonding the Rh group to the cyclosiloxane of formula (III), and provided further (a) at least one X group of Formula (III) is a —Z-R^{h} group; (b) if Z is a divalent hydrocarbon group, a=1, c=2, e+f+g+h+i +j=0 and d>0, then at least one d unit (ie. Y₂₋ₒRₒSiO_{2/2}) contain a —Z-G group or the c units (ie. Y₃₋ᵤRᵤSiO_{1/2}) have no —Z-G group or at least two —Z-G groups, (c) if Z is a divalent hydrocarbon group, a=1,c=2 and d+e+f+g+h+i +j =0, then the c units (ie. Y₃₋ᵤRᵤSiO_{1/2}) have no —Z-G group or at least two —Z-G groups, and (d) if g+h+i+j>0 then c+d+e+f>0.

For example, organopolysiloxane B according to Formula III may be the compound of Formula V, where Me is methyl, d is an average of 8 and x is an integer from 1 to 15.

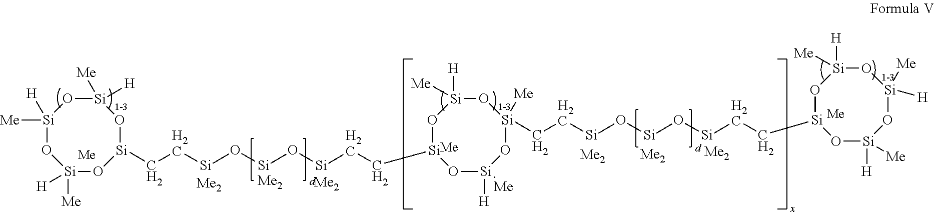

Further details of organopolysiloxanes according to Formula III and that comprise of from 0.25% to 1% silicon-bonded hydrogen atoms by weight are described in International Patent Publication No. WO2003/093349, each of which may be organopolysiloxane B of the present invention, such that the viscosity of Organopolysiloxane B≦100 mm²/s. Consequently, the disclosure of such organopolysiloxanes described in International Patent Publication No. WO2003/093349 is incorporated herein by reference.

In order to enhance the mixing or organopolysiloxane A and B, the organopolysiloxanes may be liquid at standard ambient temperature and standard atmospheric pressure. Organopolysiloxane A may have a viscosity ≦400 mm²/s. Organopolysiloxane B may have a viscosity ≦100 mm²/s. Selecting organopolysiloxanes with the appropriate viscosity enables the mixing to occur without the need for solvents for the organopolysiloxanes. Consequently, the step of mixing organopolysiloxane A and/or B with a solvent may be absent from the method of the present invention.

The molar ratio of the alkenyl groups of organopolysiloxane A to the silicon-bonded hydrogen atoms of organopolysiloxane B in the mixture may typically be of from 0.5:1.5 to 1.5:0.5, alternatively 1:1. A slight excess of either one of organopolysiloxane A or organopolysiloxane B, that is, within the molar ratio of from 0.5:1.5 to 1.5:0.5, provides for a reaction producing a satisfactory elastomeric silicone powder.

Organopolysiloxane A and organopolysiloxane B may be mixed in step (i) prior to step (ii). This may be achieved by mixing in a batch reactor, or continuously, for example in a static mixer or in a dynamic mixer, or directly in a nozzle, such as in a mixing nozzle. Batch reactor mixing and continuous mixing are known mixing techniques in the art. After mixing, the mixture is then fed to the spray curing tank via a nozzle (e.g. rotary nozzle) where step (ii) takes place. When the mixing takes place in a mixing nozzle, the two organopolysiloxanes are fed to the nozzle separately, but are mixed in the nozzle before the mixture is sprayed into the spray curing chamber during step (ii).

Alternatively, the mixing of organopolysiloxane A and organopolysiloxane B in step (i) occurs simultaneously with the spray curing of step (ii). For example, a 2-fluid nozzle may transmit the organopolysiloxanes to the spray curing chamber simultaneously but in separate conduits (ie co-spraying), thereby only permitting mixing of the organopolysiloxanes during step (ii). Other appropriate nozzles may be used such as rotary nozzle, pressure nozzle, trifluid nozzle, and further nozzles known in the art. Spray curing devices are known in the art, and may also be known as spray dryer devices. These devices may possess various settings, such as inlet and outlet temperatures, pressure, vacuum appliances, heat appliances, sieves, scrapers.

Step (i) may be carried out at a temperature of above 5° C., optionally from 5 to 100° C., alternatively from 15 to 90° C., alternatively from 15 to 70° C.

In order to reduce the potential for organopolysiloxane A and B reacting prior to their release into the spray curing chamber, when step (i) and (ii) are not simultaneous, step (i) may include the addition of an inhibitor. The inhibitor may be added to organopolysiloxane A prior to mixing this pre-mixture with organopolysiloxane B. Alternatively, the inhibitor may be added to organopolysiloxane B prior to mixing this pre-mixture with organopolysiloxane A. Alternatively, organopolysiloxane A, organopolysiloxane B and the inhibitor may be simultaneously mixed together.

The inhibitor may be selected from addition-reaction inhibitors. Addition-reaction inhibitors may be any one or more of : hydrazines, triazoles, phosphines, mercaptans, organic nitrogen compounds, acetylenic alcohols, silylated acetylenic alcohols, maleates, fumarates, ethylenically or aromatically unsaturated amides, ethylenically unsaturated isocyanates, olefinic siloxanes, unsaturated hydrocarbon monoesters and diesters, conjugated ene-ynes, hydroperoxides, nitriles, diaziridines and mixtures thereof. The inhibitor may be added in the range of from 10 to 50,000 weight-ppm (parts per million) in the mixture of organopolysiloxane A and B.

The method may include the step of adding a catalyst during step (i). The catalyst may be added to organopolysiloxane A or B, or to the mixture thereof. The catalyst may be added after the addition of the inhibitor. The catalyst may be added to organopolysiloxane A, and the inhibitor to organopolysiloxane B, or inversely.

The catalyst may be selected from the platinum group metals, or transition metals, of the periodic table of the elements, such as platinum, ruthenium, rhodium, palladium, osmium and iridium, and compounds thereof, and mixtures thereof. Specific examples of these catalysts include those based on platinum such as chloroplatinic acid, chloroplatinic acid dissolved in an alcohol or a ketone and these solutions which have been ripened, chloroplatinic acid-olefin complexes, chloroplatinic acid-alkenylsiloxane complexes, chloroplatinic acid-diketone complexes, platinum black, and platinum supported on a carrier. The catalyst is to be added in a quantity sufficient to enable curing of the mixture during step (ii). For example, the catalyst may be added in a quantity that provides of from 0.1 to 1,000 weight-ppm, alternatively of from 1 to 500 weight-ppm, alternatively of from 40 to 250 weight-ppm, of platinum metal in the catalyst based on the total weight of organopolysiloxane A and B.

The method may include the step of adding a non-reactive ingredient during step (i) and/or step (ii) and/or after step (ii). When the non reactive ingredient is added after step (ii), this may be applied as a surface treatment, or modification, of the surface of the particles of the elastomeric silicone powder, for example by spray coating or spray drying. The non reactive ingredient may be a coating or personal care ingredient which possesses properties that improve the suitability of the elastomeric silicone powder for use in a coating composition or in a personal care composition, whilst also not inhibiting the curing of organopolysiloxane A and B or evaporating during step (ii).

The non reactive coating or personal care ingredient may be added in the form of a liquid or a solid. Liquid forms include emulsions, dispersions, and solutions. Solid forms include powders or granules.

The non-reactive personal care ingredient may confer additional properties to the elastomeric silicone powders such as humectancy, compatibility, hydrophilicity, flow, compaction, colour, sun protection and/or moisturization.

Examples of non-reactive personal care ingredients include humectants (such as glycerin or sorbitol); powders or fillers (such as kaolin, talc, silica, clay); pigments (such as iron oxide, titanium dioxide, carbon black); colorants (such as D&C Red 33, FD&C Blue 1, FD&C Yellow 6, D&C Yellow 10, ultramarines, henna, indigo); hair dyes (such as 5-amino-2,6-dimethoxy-3-hydroxypyridine; 2-amino-4-hydroxyethylaminoanisole; 2-amino-4-hydroxyethylaminoanisole sulfate; 4-amino-3-nitrophenol; 2-amino-4-nitrophenol sulfate; 4,6-bis(2-hydroxyethoxy)-m-phenylenediamine HCl; 2-chloro-5-nitro-N-hydroxyethyl p-phenylenediamine; 2-chloro-p-phenylenediamine; 2,6-diaminopyridine; dihydroxyindole); vitamins (such as retinol, tocopherol, ascorbic acid, ergocalciferol, riboflavin); vitamin derivatives (such as retinyl palmitate, tocopheryl acetate); enzymes (such as proteases like papain or bromelain, lysozymes); skin bleaching agents (such as hydroquinone, arbutin, kojic acid, niacinamide, alpha hydroxyl acids); preservatives (such as phenoxyethanol, bronopol, iodopropynyl butylcarbamate, benzyl alcohol, DMDM hydantoin, methyl paraben, ethyl paraben, propyl paraben, butyl paraben, pentylene glycol, methylisothiazolinone); antimicrobials (such as zinc pyrithione, silver and its derivatives, chlorohexidine digluconate, essential oils, triclosan); antioxidants (such as epigallocatechin gallate, resveratrol, butylhydroxytoluene, butylated hydroxyanisole, t-butyl hydroquinone, zinc dibutyldithiocarbamate, chitosan salicylate); pH controlling agents (such as carboxylic acid, hydrochloric acid, acetic acid, lactic acid, citric acid); fragrances (such as hexyl cinnamic aldehyde, dodecalactone gamma, patchouli, olibanum resinoid, labdanum, vetivert, copaiba balsam, geraniol, geranyl acetate, linalool, citronellol, terpinyl acetate, benzyl salicylate, musk fragrances, macrocyclic ketones); plasticizers (such as acetyl triethyl citrate, triacetin, sucrose acetate isobutyrate, trimethyl pentanyl diisobutyrate, castor isostearate succinate, isoeicosane); film formers (such as cellulose acetate butyrate, cellulose acetate propionate, polyester-5); sunfilters (such as ethyl hexyl methoxycinnamate, octyl salicylate, glyceryl aminobenzoate, menthyl anthranilate, octocrylene, benzophenone 1-12, 3-benzylidene camphor, octyl triazone, PABA, titanium dioxide, VA/crotonates/methacryloxybenzophenone-1 copolymer). Further personal care ingredients are known by the skilled in the art and will be chosen upon need and desired benefit.

The level of non-reactive ingredient in the powder will vary based on the final use of the elastomeric silicone powder and the type of ingredient. The non reactive personal care ingredient may be present at a level ranging of from 0 to 50, 0 to 30, or 5 to 15% wt of the total weight of the final elastomeric silicone powder. When glycerin is used as the non reactive personal care ingredient, it may be included at 30% wt of the final elastomeric silicone powder.

The non-reactive coating ingredient may confer additional properties to the elastomeric silicone powders such as matting effect, anti slip, soft feel, structure effect, anti-scratch, anti-white mark, anti-squeak, anti-abrasion and anti-blocking effect.

Examples of non reactive coating ingredients include adhesives (such as polyacrylics, polyesters, epoxys, polyurethanes, polyurethane/acrylic copolymers, polyacrylates, polyimides, polycyanoacrylates, polyamides, polychloroprene, poly(vinyl alcohol), poly(ethylene/vinyl acetate), hydroxyl-terminated polybutadiene); wetting additives (such as silicone polyethers, ethoxylated acetylinic diols, fluoro surfactants); flow and leveling additives (such as silicone polyethers, acrylates); slip additives (such as polydimethylsiloxanes, silicone polyethers, organic waxes, synthetic waxes); foam control additives (silicone emulsions, silicone polyethers, mineral oils, polyether derivate of fatty acid); fillers (such as fumed silica, precipitated silica, fused silica, quartz powder, clay, wollastonite; aluminosilicates, such as feldspars; silicates, such as kaolin, talc, mica, magnesite; alkaline earth metal carbonates, such as calcium carbonate, magnesium carbonate, dolomite; alkaline earth metal sulfates, such as calcium sulfate, gypsum; and mixtures thereof); fungicides (such as mepronil, thifluzamide, pencycuron, biphenyl, tianidil, kasugamycin, streptomycin, carpropamid, pyraclostrobin); flame retardants (such as clay, polybrominated diphenyl ethers, polybrominated biphenyls, chloroparaffins, melamine, urea, tris(chloroethyl)phosphate, tricresyl phosphate, resorcinol bis(diphenylphosphate), aluminium hydroxide, magnesium hydroxide, ammonium sulfate, aluminium phosphate, antimony trioxide, zinc borates, slaked lime); pigments (such as inorganic white pigments, such as titanium dioxide, barium sulfate, zinc oxide, zinc sulfide, basic lead carbonate, antimony trioxide, lithopone (zinc sulfide+barium sulfate); inorganic colored pigments, such as iron oxides, carbon black, graphite, zinc yellow, zinc green, ultramarine, manganese black, antimony black, manganese violet, Paris blue, Paris green; organic colored pigments, such as sepia, gamboge, Cassel brown, toluidine red, paranitraniline red, Hansa yellow, indigo, azo dyes, anthraquinoid and indigoid dyes and dioxazine, quinacridone, phthalocyanine, isoindolinone and metal complex pigments); thickeners (such as xanthanes; acrylate thickeners; associative thickeners, such as polyurethane thickeners; cellulose ethers such as methyl- or hydroxyethylcellulose, methylhydroxypropylcellulose); antioxidants (such as alkylated monophenols alkylthiomethylphenols, hydroquinones and alkylated hydroquinones, tocopherols, hydroxylated thiodiphenyl ethers, hydroxybenzylated malonates, aromatic hydroxybenzyl compounds and/or triazine compounds); heat stabilizers (such as tin stabilizers, mixed metal stabilizers); plasticizers (such as those based on polycarboxylic ethers, melamine, naphthalene, polyether derivatives); dispersing agents (such as metal salts of polyacrylic acid, metal salts of carboxylic acid copolymer).

Further coating ingredients, among which antimicrobials, preservatives, lubricants, rheology additives, optical brighteners, anticaking agents, antistatic agents and blowing agents, are known by the skilled in the art and will be chosen upon need and desired benefit.

The non reactive coating ingredient may be present at a level ranging of from 0 to 50, 0 to 30, or 5 to 15% wt of the total weight of the final elastomeric silicone powder.

The step of curing the mixture in step (ii) may be achieved by a condensation reaction, e.g. a hydrosilylation reaction. The temperature at which the step of curing occurs (i.e. the temperature at the exit of the nozzle into the spray curing chamber in which step (ii) occurs) is of from 80 to 300° C., alternatively of from 100 to 250° C., alternatively of from 100 to 200° C.

The method may further include step (iii), wherein the elastomeric silicone powder produced during step (ii) is collected from the spray curing chamber in which step (ii) is carried out. Step (iii) may be carried out in a cyclone. Filters may also be used in step (iii) in order to collect powders of the desired size and/or weight.

The method may be solventless or may further include step (iv), wherein the elastomeric silicone powder may be dispersed in a solvent or a mixture of solvents. Examples of solvents include organic solvents (such as aliphatic compounds (hexane, mineral oil, isododecane, (iso-)paraffins); aromatic compounds (toluene, xylene, benzene); mineral spirits; methyl ethyl ketone; n-butyl acetate; t-butyl alcohol; ethylene glycol; vegetable oils (castor oil, sunflower oil, olive oil, coconut oil, rice bran oil); alcohols (ethanol, isopropanol)); silicone oils (such as cyclosiloxanes, dimethicones of viscosities of from 0.5 to 10,000 mm²/s, caprylyl methicone); water.

In a second aspect of the present invention, there is provided any of the mixtures of organopolysiloxane A and organopolysiloxane B, optionally with the inhibitor, catalyst, non reactive personal care or coating ingredient, described in the first aspect of the present invention for spray curing in the methods of the first aspect of the present invention so as to form an elastomeric silicone powder.

In a third aspect of the present invention, there is provided an elastomeric silicone powder prepared according to any of the methods of the first aspect of the present invention.

The elastomeric silicone powder may have application in personal care compositions. Personal care compositions include those compositions which may be used in the care of keratinous substrates such as skin, hair, nails and lashes. Consequently, in a fourth aspect of the present invention there is provided a use of the elastomeric silicone powder of the first aspect of the present invention for personal care.

In a fifth aspect of the present invention, there is provided a personal care composition comprising the silicone powders of the first aspect of the present invention. The personal care composition may comprise from 0.1 to 99.9% wt of the total weight of the personal care composition, alternatively from 1 to 85% wt, alternatively from 1 to 25% wt, elastomeric silicone powder according to the first aspect of the present invention. When used for the care of skin, the personal care composition may be in the form of a lotion, cream, mask, deodorant, antiperspirant, foundation, lipstick, blush, ointment. When used for the care of hair, the personal care composition may be in the form of a shampoo, balm, conditioner, setting or fixative product. When used for the care of nails, the personal care composition may be in the form of a varnish or a coating. When used for the care of lashes, the personal care composition may be in the form of a mascara.

The benefit of using such personal care compositions comprising the elastomeric silicone powder of the present invention in the care of skin may be feel improvement, soft focus, for moisturization, sun protection. The benefit of using such personal care compositions comprising the elastomeric silicone powder of the present invention in the care of hair may be for sebum absorption or ease of brushing out from a dry shampoo.

Coating compositions include those compositions which may be used to modify or treat the surface of materials such as wood, plastic, leather, electronic devices, paper or metal. The elastomeric silicone powder may be included in such coating compositions. Consequently, in a sixth aspect of the present invention, there is provided a use of the elastomeric silicone powder of the first aspect of the present invention for use in a coating composition.

In a seventh aspect of the present invention there is provided a coating composition comprising the silicone powders of the first aspect of the present invention. The coating composition may comprise from 1 to 20% wt of the total weight of the coating composition, alternatively from 5 to 15% wt, alternatively from 8 to 12% wt, elastomeric silicone powder according to the first aspect of the present invention. Coating compositions may be in the form of inks, over print varnishes, lacquers/clear coats and pigmented paints. These may be solventless, solvent borne or waterborne and may be air drying, heat cured or radiation cured.

The benefit of using such coating compositions comprising the elastomeric silicone powder of the present invention may be for mattifying, for imparting scratch resistance of wood flooring and laminates, for feel modification, for reduced dirt pick up, for imparting antifouling properties (lotus effect); in leather treatment for example for mattifying and anti wear.

As described above, the method of the present invention may be characterized by the amount of deposit of elastomeric silicone on the walls of the spray curing chamber, i.e. the amount of product that ends up cured and stuck to the walls of the spray curing chamber and that is not in loose powder form. This deposit will typically not be removed by standard equipments such as air bloom, air brush, air shock blower, but instead will typically require manual scraping to be removed. The skilled person would be aware of how to identify such an amount. However, for the avoidance of doubt, the amount of deposit may be measured by removing the loose powder from the walls of the chamber (e.g. by the use of an air brush), then scraping the walls of the chamber following step (ii) and the removal of loose powder, weighing the amount collected from the scraping, and calculating the amount collected as a % of the total produced based on analysis of the amount of ingredients in the mixture of step (i).

As described above, the method or the elastomeric silicone powder of the present invention may be characterized by the size of particle or agglomerate produced by the method. The skilled person would be well aware how to analyse the size of particles or agglomerates. However, for the avoidance of doubt, the size may be analysed by visual analysis using a microscope. More particularly, several (eg 5 or more) random images of the collected elastomeric silicone powder may be viewed at the same magnification via a microscope and the diameter of the particles or agglomerates in each view determined. A mean of all determined diameters is then calculated and may be used to characterize the size of the elastomeric silicone powder.

A particle that is substantially spherical is one in which all diameters across the central point of the particle deviate by no more than 20%, alternatively not more than 10%, alternatively not more than 5%.

Various methods exist which may be used to quantify the levels of silicon bonded hydrogen atoms or alkenyl groups.

Such methods or techniques include, but are not limited to, titration, infra-red, near infrared (FT-NIR), volumetry of hydrogen gas, and nuclear magnetic resonance (NMR).

29Si NMR spectroscopy may be used to determine the content of silicon bonded hydrogen atoms or alkenyl substitution (in weight %). This method allows distinguishing between all species bearing Si atoms, therefore including species with silicone bonded hydrogen and alkenyl functionalities bonded to a silicon atom. The sample (40 vol %) needs to be dissolved in deutero-chloroform (60 vol %), which contains chromium acetyl acetonate (0.05M concentration in the solvent). The solution can be measured in a 10 mm NMR tube in an NMR spectrometer (400 MHz). The method is called “29Si inverse gated” to allow quantitative conditions for the measurement. Parameters include a spectral width of 250 ppm, a pulse length of the 90°, a relaxation delay of 15 s, and a number of scans of 2600. After the measurement, the free induction decay is Fourier-transformed into the frequency spectrum, tetramethyl silane is used as calibration standard (0 ppm alkenyl group or silicon-bonded hydrogen atom). The peaks need to be integrated, made in relation to the mass unit of the corresponding species and the content of silicone bonded hydrogen or alkenyl functionalities bonded to a silicon atom may be calculated. The method may be applicable for all types of silicones containing methyl as principal organic functionality, and may be adapted for the calculation when other alkyl groups or functional groups are present.

FT-NIR method may be used to quantify the percentage of alkenyl groups by weight of organopolysiloxane A, by comparing the absorbance of a specific band in an infrared spectrum with the absorbance of the same band in a reference spectrum of known concentration, in the present case, the band corresponding to the carbon-carbon double bond of the alkenyl functionality. This procedure may be based on ASTM E-168 (Standard Practices for General Techniques of Infrared Quantitative Analysis) where the material to be analysed is diluted in an appropriate solvent of spectrophotometric grade. The dissolution may require mixing and may take several hours to complete. Standards of known concentrations are used as comparison for the quantification of the alkenyl functionality.

A bromine titration method may be used to quantify the percentage of silicon-bonded hydrogen atoms by weight of organopolysiloxane B, according to an estimated SiH content.

### EXAMPLES

The Examples and Comparative Examples, of which compositions are given in Table 1, were prepared as follows:
Organopolysiloxane A is put in a beaker
the inhibitor is added
Organopolysiloxane B is added
the catalyst is added
the optional other ingredient is added
the mixture is let in the spray curing pipes and nozzle and led to the spray curing tank to react and cure in the form of an elastomeric silicone powder.

The mixtures were prepared at room temperature (20-25° C.). The inhibitor consisted of ethylene cyclohexanol, the catalyst consisted of a platinum complex.

The spray curing tank was a MOBILE MINOR™ Spray dryer from GEA Process Engineering Inc., with the following settings: an inlet temperature of 250-300° C., an outlet temperature of 130-150° C., a nozzle pressure of 1.5 Bar.

TABLE 1

| | | | | | Inhibitor | Catalyst | Other | Deposit |
|---|---|---|---|---|---|---|---|---|
| | Organopolysiloxane A | g | Organopolysiloxane B | g | (g) | (ppm Pt) | ingredient | (%) |
| Comparative | linear vinyl terminated | 200.00 | linear | 2.55 | 0.06 | 10 | — | 9.0 |
| Example 1 | polymer having an alkenyl | | hydrogenpolysiloxane | | | | | |
| | content of 0.47% and | | (pendant) of H content of | | | | | |
| | viscosity of 430 | | 1.5% and viscosity of 30 | | | | | |
| | mm2/s (M^{Vi}D₁₅₀M^{Vi}) | | mm²/s (MD^{H}₆₅M) | | | | | |
| Comparative | linear vinyl terminated | 200.00 | branched | 4.51 | 0.06 | 10 | — | 5.0 |
| Example 2 | polymer having an alkenyl | | hydrogenpolysiloxane | | | | | |
| | content of 0.47% and | | (pendant) of H content of | | | | | |
| | viscosity of 430 | | 0.85% and viscosity of 15 | | | | | |
| | mm²/s (M^{Vi}D₁₅₀M^{Vi}) | | mm²/s (MD^{H}₁₀D₃T₃M) | | | | | |
| Example 1 | linear polymer with | 200.00 | linear | 8.59 | 0.06 | 10 | — | 0 |
| | hexenyl groups in terminal | | hydrogenpolysiloxane | | | | | |
| | and pendant positions, | | (pendant) of H content of | | | | | |
| | having an alkenyl | | 0.92% and viscosity of 70 | | | | | |
| | content of 0.97% and | | mm²/s (M₅₀D₃₀M) | | | | | |
| | viscosity of 370 | | | | | | | |
| | mm²/s (M^{Hex}D₁₄₈D^{Hex}₂M^{Hex}) | | | | | | | |
| Example 2 | linear vinyl terminated | 200.00 | linear | 23.91 | 0.06 | 10 | — | 0 |
| | polymer, having an alkenyl | | hydrogenpolysiloxane | | | | | |
| | content of 2.70% and | | (pendant) of H content of | | | | | |
| | viscosity of 22 | | 0.92% and viscosity of 70 | | | | | |
| | mm²/s (M^{Vi}D₂₅M^{Vi}) | | mm²/s (MD^{H}₅₀D₃₀M) | | | | | |
| Example 3 | linear vinyl terminated | 200.00 | a linear | 11.51 | 0.06 | 10 | — | 0 |
| | polymer, having an alkenyl | | hydrogenpolysiloxane | | | | | |
| | content of 1.30% and | | (pendant) of H content of | | | | | |
| | viscosity of 75 | | 0.92% and viscosity of 70 | | | | | |
| | mm²/s (M^{Vi}D₅₀M^{Vi}) | | mm²/s (MD^{H}₅₀D₃₀M) | | | | | |
| Example 4 | Q-branched polymer with | 200.00 | linear | 10.36 | 0.06 | 5 | — | 0 |
| | vinyl groups having an | | hydrogenpolysiloxane | | | | | |
| | alkenyl content of 1.17% | | (pendant) of H content of | | | | | |
| | and viscosity of 100 | | 0.92% and viscosity of 70 | | | | | |
| | mm²/s (Q(D₃₀M^{Vi})₄) | | mm²/s (MD^{H}₅₀D₃₀M) | | | | | |
| Example 5 | Q-branched polymer with | 50.00 | linear | 3.06 | 0.015 | 5 | — | 0 |
| | vinyl groups having an | | hydrogenpolysiloxane | | | | | |
| | alkenyl content of 1.17% | | (pendant) of H content of | | | | | |
| | and viscosity of 100 | | 0.75% and viscosity of 5 | | | | | |
| | mm²/s (Q(D₃₀M^{Vi})₄) | | mm²/s (MD^{H}_{3.2}D_{5.8}M) | | | | | |
| Example 6 | Q-branched polymer with | 350.00 | linear | 18.3 | 0.084 | 5 | 40.0 g of | 0 |
| | vinyl groups having an | | hydrogenpolysiloxane | | | | dimethicone | |
| | alkenyl content of 1.17% | | (pendant) of H content of | | | | 100 mm²/s | |
| | and viscosity of 100 | | 0.92% and viscosity of 70 | | | | | |
| | mm²/s (Q(D₃₀M^{Vi})₄) | | mm²/s (MD^{H}₅₀D₃₀M) | | | | | |
| Example 7 | Q-branched polymer with | 200.00 | linear | 10.36 | 0.06 | 10 | 80.0 g of | 0 |
| | vinyl groups having an | | hydrogenpolysiloxane | | | | glycerin | |
| | alkenyl content of 1.17% | | (pendant) of H content of | | | | | |
| | and viscosity of 100 | | 0.92% and viscosity of 70 | | | | | |
| | mm²/s (Q(D₃₀M^{Vi})₄) | | mm²/s (MD^{H}₅₀D₃₀M) | | | | | |

Example 4 is a free flowing powder without agglomerates, feeling soft and cushiony.

Example 5 is a free flowing powder with little agglomerates, feeling soft, not dry and cushiony.

Example 6 is a free flowing powder with little agglomerates, which does not feel dry, but slightly cushiony.

Example 7, containing glycerin, was used in the water-in-oil creams of Examples 8 and 9. Examples 8 and 9 ingredients are listed in Table 2, and were prepared according to the following procedure: phase A ingredients were mixed until homogeneous, water was slowly added to phase A mixing until homogeneous (at 500-1000 rpm), when all water was added, mixing was pursued for an additional 5 minutes under high shear (2000 rpm). Example 8 and Example 9 achieved a viscosity of 34,400 mm²/s and 19,800 mm²/s respectively (measured with a Brookfield HRV device, spindle 5, speed 10). Examples 8 and 9, when applied to skin, showed nice feel.

Example 8 was able to demonstrate a slight wrinkle blurring effect in the crow's feet and under-eye areas, indicated by a decreased number of wrinkles using an image analysis device (Visia). 0.04g of cream were applied on a surface of between 10 to 13 cm² over the crow's feet area and the under eye, and rubbed in by the panelist himself. Once the product applied was rubbed in to the skin, pictures were taken at some time intervals-15 minutes, 1, 3, 5 hour(s). The Wrinkle Analysis performs quantitative analysis and scoring for assessing the severity of facial wrinkles and fine lines. The analysis algorithm operates on a high-resolution Visia-CR image captured in Standard II lighting modality (uniform white light).

TABLE 2

| Ingredients - W/O (% wt) | Example 8 | Example 9 |
|---|---|---|
| Phase A | | |
| PEG-10 Dimethicone | 2.00 | 2.00 |
| Cyclopentasiloxane (and) Dimethiconol | 3.50 | 3.50 |
| Pentaerythrityl Tetraisostearate | 4.10 | 4.10 |
| Dimethicone 1.5 mm²/s | 12.40 | 12.40 |
| Example 7 - glycerin 30% wt | 5.00 | 2.98 |
| Phase B | | |
| Water | 73.00 | 75.02 |

1. A method for preparing an elastomeric silicone powder comprising the steps of:
(i) forming a mixture of organopolysiloxane A and organopolysiloxane B, wherein organopolysiloxane A comprises more than 0.5% alkenyl groups by weight of the organopolysiloxane A, organopolysiloxane B comprises from 0.25_% to 1% silicon-bonded hydrogen atoms by weight of the organopolysiloxane B; and
(ii) spray curing the mixture of step (i) to form an elastomeric silicone powder.

2. The method according to claim 1, wherein a spray curing chamber is used with step (ii) and wherein less than 0.5% of the mixture formed in step (i) is deposited on walls of the spray curing chamber in which step (ii) is practiced.

3. The method as claimed in claim 1, where the molar ratio of the alkenyl groups of organopolysiloxane A to the silicon-bonded hydrogen atoms of organopolysiloxane B in the mixture is of from 0.5:1.5 to 1.5:0.5.

4. The method as claimed in claim 1, where forming the mixture of organopolysiloxane A and organopolysiloxane B in step (i) occurs simultaneously with the spray curing of step (ii).

5. The method as claimed in claim 1, wherein the elastomeric silicone powder forms particles, or agglomerates of particles, with an average diameter of 1 to 1000 μm.

6. The method as claimed in claim 1, wherein organopolysiloxane A comprises or consists of linear organopolysiloxanes.

7. The method as claimed in claim 1, wherein organopolysiloxane A comprises or consists of branched organopolysiloxanes.

8. The method as claimed in claim 1, wherein organopolysiloxane A comprises at least one Q-unit (SiO_{4/2}) and at least one alkenyl group is bound to the Q-unit.

9. The method as claimed in claim 1, wherein the alkenyl groups have a carbon chain length of from 2 to 6 carbon atoms.

10. The method as claimed in claim 1, wherein the organopolysiloxane A comprises less than 15% alkenyl groups by weight of the organopolysiloxane A.

11. The method as claimed in claim 1, wherein organopolysiloxane A has a viscosity of ≦400mm²/s.

12. The method as claimed in claim 1, wherein organopolysiloxane B comprises or consists of cyclic organopolysiloxanes.

13. The method as claimed in claim 1, wherein organopolysiloxane B comprises or consists of linear organopolysiloxanes.

14. The method as claimed in claim 1, wherein organopolysiloxane B comprises or consists of branched organopolysiloxanes.

15. The method as claimed in claim 1, wherein organopolysiloxane B has the general formula:
[R^{f}₂HSiO_{1/2}]_{k′[R}^{f}₃SiO_{1/2}]_{r′}[R^{f} HSiO_{2/2}]_{m′}[R^{f}₂SiO_{2/2}]_{s′}[R^{f} SiO_{3/2}]_{p′}[SiO_{4/2}]_{q′}
where each R^{f} is a univalent organic group that does not contain aliphatic unsaturation; k′≧0; r′≧0; 1≦k′+r′10; 30 ≧m′≦200; 10s′≦100; 0 ≦p′≦10; and q′=1.

16. method as claimed in claim 1, wherein organopolysiloxane B has the general formula:
R^{g}₃SiO_{1/2}((CH₃)₂SiO_{2/2})_{e′}(R^{g}₂SiO_{2/2})_{f′})SiO_{1/2}R^{g}₃
where each R^{g} is independently selected from an alkyl group having 1 to 4 carbon atoms or hydrogen, e′ is 0 or an integer, and f′ is an integer.

17. method as claimed in claim 1, wherein organopolysiloxane B comprises at least one cyclosiloxane according to formula(III): 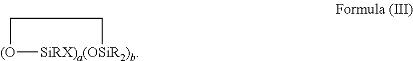
where each R is independently selected from a hydrogen atom and a monovalent hydrocarbon group comprising 1 to 20 carbon atoms which is free from aliphatic unsaturation, a is an integer from 1 to 18, b is an integer from 1 to 19, a+b is an integer from 3 to 20, each X is an independently selected functional group selected from a halogen atom, an ether group, an alkoxy group, an alkoxyether group, an acyl group, an epoxy group, an amino group, or a silyl group, or a —Z—R^{h} group, where each Z is independently selected from an oxygen and a divalent hydrocarbon group comprising 2 to 20 carbon atoms, each R^{h} group is independently selected from-SiRᵥY₃₋ᵥ, or a group described by formula (IV):
(Y₃₋ᵤRᵤSiO_{1/2})_{c}(Y₂₋ₒRₒSiO_{2/2})_{d}(Y₁₋ₚRₚSiO_{3/2})ₑ(SiO_{4/2})_{f}(CR_{q}Y_{1-q})_{g}(CRᵣY₂₋ᵣ)ₕ(O(CRₛY₂₋ₛ)ᵢ(CRₜY₃₋ₜ)ⱼ Formula (IV):
where each R is as described above, the sum of c+d+e+f+g+h+i+j is at least 2, u is an integer from 0 to 3, o is an integer from 0 to 2, p is an integer from 0 to 1, q is an integer from 0 to 1, r is an integer from 0 to 2, s is an integer from 0 to 2, t is an integer from 0 to 3, v is an integer from 0 to 3, each Y is an independently selected functional group selected from a halogen atom, an ether group, an alkoxy group, an alkoxyether group, an acyl group, an epoxy group, an amino group, a silyl group, or a Z-G group, where Z is as described above, each G is a cyclosiloxane described by formula (V): 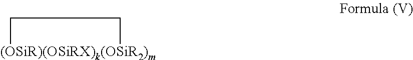
where R and X are as described above, k is an integer from 0 to 18, m is an integer from 0 to 18, k+m is an integer from 2 to 20, provided in formula (IV) that one of the Y groups is replaced by the Z group bonding the R^{h} group to the cyclosiloxane of formula (III), and provided further (a) at least one X group of formula (III) is a —Z—R^{h} group; (b) if Z is a divalent hydrocarbon group, a=1, c=2, e+f+g+h+i+j=0 and d>0, then at least one d unit (Y₂₋ₒRₒSi_{2/2}) contain a —Z-G group or the c units (Y₃₋ᵤRᵤSiO_{1/2}) have no —Z-G group or at least two —Z-G groups, (c) if Z is a divalent hydrocarbon group, a=1, c=2 and d+e+f+g+h+i+j=0, then the c units (Y₃₋ᵤRᵤSiO_{1/2}) have no —Z-G group or at least two —Z-G groups, and (d) if g+h+i+j>0 then c+d+e+f>0.

18. The method as claimed in claim 1, wherein organopolysiloxane B has a viscosity of ≦100 mm²/s.

19. The method as claimed in claim 1, wherein step (ii) cures the mixture in a hydrosilylation reaction.

20. The method as claimed in claim 1, wherein the mixture includes a platinum catalyst.

21. The method as claimed in claim 1, wherein the mixture includes a hydrosilylation inhibitor.

22. The method as claimed in claim 1, wherein step (i) is carried out at a temperature of from 5 to 100° C.

23. The method as claimed in claim 1, wherein step (ii) is carried out at a temperature of from 80 to 300° C.

24. An elastomeric silicone powder prepared according to the method as claimed in claim 1.

25. A coating composition comprising the elastomeric silicone powder prepared according to the method as claimed in claim 1.

26. (canceled)

27. A personal care composition comprising the elastomeric silicone powder prepared according to the method as claimed in claim 1.

28. (canceled)
